(19) United States
(12) Patent Application Publication
(10) Pub. No.: US 2025/0304945 A1
(43) Pub. Date: Oct. 2, 2025

(54) MICROGELS, LIVE CELL-LADEN MICROGELS, PRODUCTS HAVING SUCH MICROGELS, AND METHODS AND APPARATUSES FOR FABRICATING SUCH MICROGELS VIA PHOTOPOLYMERIZED IN-AIR DROP ENCAPSULATION

(71) Applicant: Lawrence Livermore National Security, LLC, Livermore, CA (US)

(72) Inventors: Elisa Marie Wasson, Livermore, CA (US); Michael Gregory Triplett, Pleasanton, CA (US); Congwang Ye, San Ramon, CA (US); Jesse Thomas Ahiquist, San Jose, CA (US)

(21) Appl. No.: 19/093,095

(22) Filed: Mar. 27, 2025

Related U.S. Application Data
(60) Provisional application No. 63/572,828, filed on Apr. 1, 2024.

(51) Int. Cl. C12N 11/02; A61L 27/52

(52) U.S. Cl. CPC C12N 11/02; A61L 27/52

(57) ABSTRACT

A live cell-laden microgel, in accordance with one aspect of the present invention, includes at least one live cell and a photocured resin. An in-air drop formed microgel, in accordance with one aspect of the present invention, includes a component selected from the group consisting of DNA, RNA, a protein, a peptide, an antibody, a living-cell produced catalyst, a nucleic acid-based material, a prokaryotic cell, an eukaryotic cell, and a different type of biological component.

### RELATED APPLICATION

This application claims priority to U.S. Provisional Patent Appl. No. 63/572,828, filed Apr. 1, 2024, and which is incorporated by reference.

This invention was made with Government support under Contract No. DE-AC52-07NA27344 awarded by the United States Department of Energy. The Government has certain rights in the invention.

### FIELD OF THE INVENTION

The present invention relates to microgels and related products, and more particularly, this invention relates to live cell-laden microgels, products having such microgels, and methods and apparatuses for fabricating such microgels via photopolymerized in-air drop encapsulation.

### BACKGROUND

Cell encapsulation to date has mainly consisted of bulk emulsification techniques or microfluidic encapsulation of cells in either aqueous droplets or hydrogel beads, otherwise known as microgels. However, such approaches traditionally require an immiscible oil phase, as shown in FIG. 10. The oil phase does not support cell viability long term, therefore either limiting the duration of cell culture in the case of aqueous droplets or requiring its removal in the case of microgels. Removing the oil phase to a degree sufficient for long term cell culture can be challenging, requiring the use of harsh solvents or extensive washing steps, which results in a large amount of contaminant inherently being present in the formed droplets or beads, as well as a loss of 75% or more of the materials being processed. Moreover, the oil phase and/or processing to separate the oil phase may lead to a reduction in cell viability. While microfluidic devices enable the production of microgels with highly controlled size and monodispersity, throughput of these devices is limited due to limitations in flow rate (e.g., 1-10 μL/min) requiring the use of multiple devices in parallel to scale up fabrication of droplets and microgels. As a result, such processes are not readily scalable and may be difficult to translate into clinical, pharmaceutical, or industrial processes.

To circumvent these challenges, in-air encapsulation systems have been developed and are capable of generating microgels with tunable size at throughputs two orders of magnitude higher than droplet microfluidics. This technique fabricates microdroplets in the air by generating instabilities in a liquid jet ejected from a nozzle through vibration of the nozzle. However, many current in-air systems rely on enzymatic or ionic crosslinking of microgels limiting the types of materials that can be used. Using photopolymerizable materials offers more flexibility in material choice and rapid curing, yet fabricating microgels with photocurable materials using commercially available in-air systems requires forming a secondary shell therefore limiting scalability.

### BRIEF SUMMARY

A live cell-laden microgel, in accordance with one aspect of the present invention, includes at least one live cell and a photocured resin.

An in-air drop formed microgel, in accordance with one aspect of the present invention, includes a component selected from the group consisting of DNA, RNA, a protein, a peptide, an antibody, a living-cell produced catalyst, a nucleic acid-based material, a prokaryotic cell, an eukaryotic cell, and a different type of biological component.

A product, in accordance with various approaches, includes a plurality of the aforementioned microgels formed into a predefined shape.

Other aspects and advantages of the present invention will become apparent from the following detailed description, which, when taken in conjunction with the drawings, illustrate by way of example the principles of the invention.

### BRIEF DESCRIPTION OF THE DRAWINGS

FIG. 1 is representation of photopolymerized in-air drop encapsulation (PIADE or photopolymerized IADE) system in operation, in accordance with one aspect of the present invention.

FIG. 2 is representation of a PIADE system in operation, in accordance with one aspect of the present invention.

FIG. 3, part A, is an image of microgels having 4T1 mammary carcinoma cells, after one day of encapsulation, where the microgels were formed via a PIADE process during experimentation to study mammalian cell viability.

FIG. 3, part B, is a chart depicting the viability of live cells in microgels formed during the experiment with different UV lamp intensities.

FIG. 4 is a diagram of a PIADE system in accordance with one approach.

FIG. 5 is a flow diagram of a method for forming composite structures from microgels, in accordance with one aspect of the invention.

FIG. 6 is a graphical depiction of a method for assembling microgels layer-by-layer to make a porous gradient tissue scaffold, in accordance with an experiment conducted in according to one aspect of the invention.

FIG. 7 includes images of the stacked and coupled microgels formed during the experiment of FIG. 6.

FIG. 8 is a system diagram of a PIADE system in accordance with one approach.

FIG. 9 is a system diagram of a PIADE system in accordance with one approach.

FIG. 10 is a depiction of formation of microgels using an immiscible oil phase in a microfluidic device.

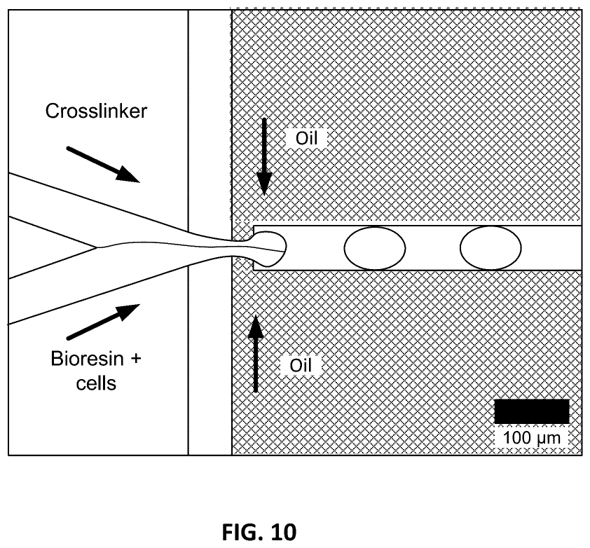

FIG. 11 is a photograph of droplets passing from the nozzle of a droplet formation portion, in accordance with one approach.

FIG. 12 is a depiction demonstrating the ability to create microgels of different cell loading in accordance with various approaches.

FIG. 13 is a representation of formation of a microgel having a live cell therein, and microgels being assembled together in a scaffold that mimics the physical characteristics of the layers of cartilage depicted adjacent thereto.

FIG. 14 includes front and side views of a PIADE system in accordance with one approach.

FIG. 15 depicts experimental results demonstrating the effect of vibration frequency on microgel diameter using the same fluid.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

Described herein is an innovative in-air photopolymerization system which improves greatly on microgel throughput over microfluidic synthesis approaches. The system, in some approaches, fabricates microdroplets in the air by generating instabilities in a liquid jet ejected from a nozzle through vibration. The droplets are then photopolymerized mid-air, achieving droplet formation up to two orders of magnitude faster than traditional microfluidic encapsulation and without the need for a secondary shell, unlike other commercially available in-air systems that use photocurable materials. To the best of the inventors' knowledge, this is the first demonstration of fabricating cell-laden microgels using the in-air photopolymerization technique.

Fabrication of live cell laden microgels using the techniques described herein may enable the generation of tissues with gradients in mechanical and chemical properties, such as for articular cartilage. Articular cartilage is essentially impossible to repair once damaged, resulting in approximately $21 billion spent annually on disability and total joint replacements in the United States. To date, engineering approaches have failed to adequately recapitulate tissue gradients. While some techniques, such as bioprinting, enable a high degree of spatial control, introducing biophysical gradients into polymer materials remains an open challenge, which can only be partially addressed through use of multiple nozzles or exchange of materials mid-print. As a result, tissue-engineered constructs available today remain monolithic or very simple, and as a result fail as in vitro screening systems and in vivo replacement tissues. Microgel based tissue scaffolds offer a promising avenue for generating complex biomimetic tissue scaffolds in a modular fashion. Microgels with tunable size, mechanical stiffness, and biochemical properties serve as individual building blocks to fabricate porous tissue scaffolds that can mimic the heterogeneity of biological tissues. In this invention we demonstrate cell encapsulation in microgels using the present in-air photopolymerization system. Additionally, in some approaches, microgels may be assembled to form a two-layered, three-layered (or more) porous scaffold with a gradient in stiffness. The innovations disclosed herein and the resulting products may be used for a wide range of applications beyond tissue engineering, such as biosensing, bioremediation, and biotherapeutic production.

The following disclosure describes new types of microgels, as well as methodology for high-throughput, tunable, cell-laden microgel production leveraging photopolymerized in-air drop encapsulation (PIADE) techniques. Presented herein is a description of the first demonstration of live cell encapsulation using PIADE that cures microgels in a gaseous environment (e.g., in common air, or some other gas or gas mixture) using light, e.g., UV light.

Production of new types of engineered biocompatible microgels of varying material composition and stiffness via PIADE fabrication processes is enabled by the teachings herein. Moreover, unique PIADE apparatuses particularly useful for encapsulation of live cells in microgels are disclosed herein.

Also presented herein is the first demonstration of gradient tissue scaffolds formed using PIADE-fabricated microgels. Cell-laden microgels of varying stiffness and material composition may be stacked layer-by-layer and unified (e.g., crosslinked) to generate gradient tissue scaffolds of a variety of desired configurations, e.g., gradients in composition, porosity, stiffness, etc. Cell laden microgels provide a direct path toward engineering tissue biophysical gradients as individual microgels can be customized, serving as building blocks that can be assembled to fabricate multi-material, multi-stiffness, multi-cellular scaffolds with tunable pore size.

The methodology presented herein enables introduction of more complexity into tissue scaffolds than has heretofore been possible by generating gradients in materials (e.g., composition), porosity, cell types, growth factors, etc. as encapsulated cargo. Moreover, microgel material and size can be easily tuned.

Other aspects of the present disclosure provide the framework to fabricate customizable living microscale constructs that can be readily translated for use in applications ranging from biosensing and bioremediation, to biofuel and biotherapeutic production.

Methodology described herein, according to various approaches, includes a PIADE-based approach that provides a high-throughput, versatile, and facile bottom-up tissue engineering method to engineer gradient tissues using cell-embedded microgels as building blocks. Disclosed herein are materials and PIADE parameters that enable embedding of live mammalian cells in biocompatible microgels for the first time and also techniques for fabricating tissue constructs possessing gradients in stiffness.

The techniques presented herein enable the first ever engineered gradient tissue using PIADE fabricated cell-embedded microgels. Cell-embedded microgels provide a direct path toward engineering tissue biophysical gradients, as individual microgels can be customized, serving as building blocks that can be assembled to fabricate multi-material, multi-stiffness, multi-cellular scaffolds with tunable pore size.

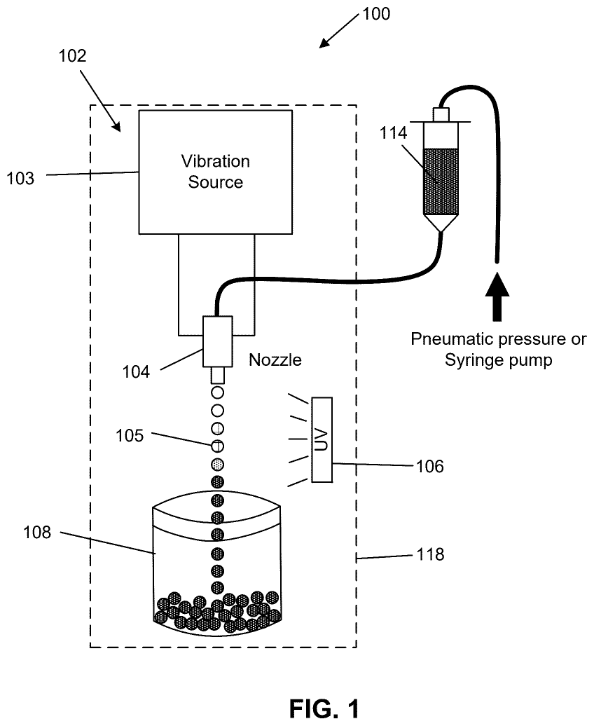

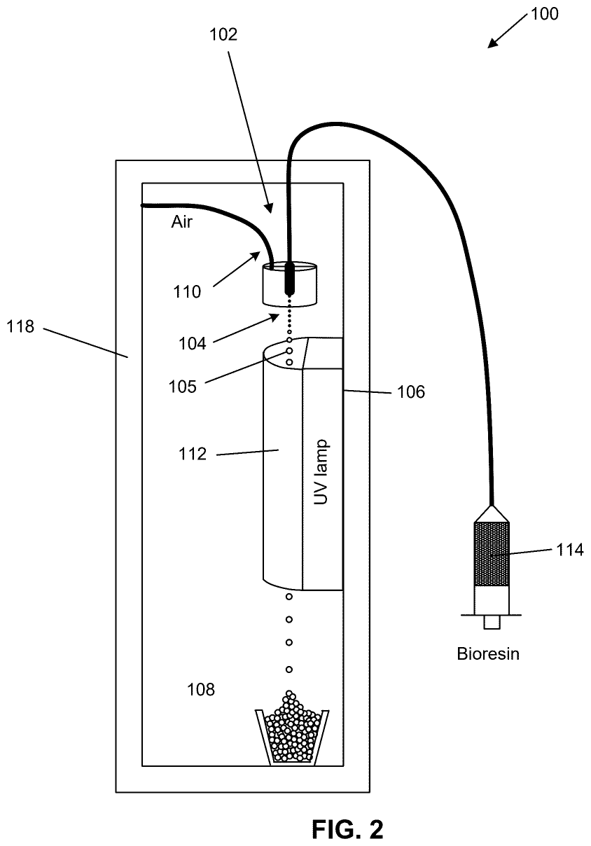

FIGS. 1-2 are representations of a PIADE system 100 in operation, in accordance with two general approaches. As an option, the present system 100 may be implemented in conjunction with features from any other embodiment listed herein, such as those described with reference to the other FIGS. Of course, however, such system 100 and others presented herein may be used in various applications and/or in permutations which may or may not be specifically described in the illustrative embodiments listed herein. Further, the system 100 presented herein may be used in any desired environment.

As shown, the system 100 includes a droplet formation portion 102, that in this configuration includes a vibration source 103 and a nozzle 104. The nozzle may include a pneumatic or jet valve, or have any suitable configuration, as would become apparent to one skilled in the art after reading the present disclosure.

FIG. 2 depicts a variation on the PIADE system 100, in which the droplet formation portion 102 includes an aerodynamically shearing gas jet 110, e.g., where the gas is air or some other suitable gas that would become apparent to one skilled in the art after reading the present disclosure. Also, a reflector 112 may be present to reflect light from the light source 106 back onto the droplets.

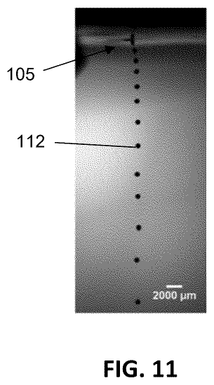

With continued reference to the system 100 of FIGS. 1-2, the droplet formation portion 102 fabricates micron-scale droplets 105 which are then crosslinked in-air (typically in milliseconds) using a light source 106 before collection, e.g., in a receptacle 108 (e.g., solid container), a liquid suspension, or an air flow. FIG. 11 is a photograph of droplets 105 passing from the nozzle of a droplet formation portion 102, in accordance with one approach.

In one approach, a receptacle 108 may contain a solution that assists in capturing the microgels, maintaining viability of the cell embedded microgels, etc. by forming a liquid suspension of the solution and microgels. In further approaches, an enclosure 118 may enclose portions of the system 100. The atmosphere within the enclosure 118 may be common air. In other approaches, the atmosphere in the enclosure may be a particular gas or gas mixture, e.g., to improve viability of live cells in the droplets and/or microgels. Accordingly, while much of the present description refers to forming microgels in air, it should be kept in mind that this is intended to equivalently encompass approaches that use gases and/or gas mixtures that are different than common air.

The mixture 114 from which the droplets 105 are formed is urged through the nozzle 104, e.g., using a pressure pump, a syringe pump, or the like. The flow rate or pressure of the fluid may be increased until the resin-cell mixture forms a stable jet at the nozzle 104. An aerodynamically shearing air jet and/or acoustic vibration force causes droplet formation. The droplets are then crosslinked in-air (typically in milliseconds) using a UV light source before collection. The cure time can be modified/selected to improve microgel production, modulate microgel properties, and/or enhance cell viability.

In accordance with various aspects of the present invention, the PIADE systems 100 disclosed herein may provide one or more, and preferably all, of the following benefits: produce microgels approximately two orders of magnitude (100×) faster than other techniques such as microfluidic oil-based encapsulation systems; decrease material loss from 80% to less than about 5%; process biomaterials that are at least 10× more viscous, and in some cases at least 100× more viscous, than mixtures usable with microfluidic oil-based encapsulation systems; more scalable, versatile and robust crosslinking than enzymatic or ionic crosslinking.

In one example of use, live cells are mixed with a photocurable resin (polymer+photoinitiator) at the desired concentration, loaded into a syringe and pumped through a nozzle, e.g., using a pressure pump, a syringe pump, or the like. The flow rate or pressure of the fluid is increased until the resin-cell mixture forms a stable jet at the nozzle. The nozzle size, flow rate of the resin-cell mixture, frequency of vibration, and cell density can all be selected (tuned) to produce microgels of varying size and number of encapsulated cells. Crosslinked cell laden microgels are then collected in a sterile mixing bath of cell culture media and subsequently dispersed into tissue culture well plates. The culture plates are then kept in a cell culture incubator to monitor growth or to be used for stacking and crosslinking into a tissue scaffold.

In preferred aspects, PIADE systems and corresponding apparatuses for forming the microgels disclosed herein are based on the systems described in U.S. Pat. No. 11,173,461, which is herein incorporated by reference. Said patent refers to “particles” and the like. Moreover, PIADE systems and corresponding apparatuses for forming the microgels disclosed herein may also and/or alternatively incorporate features from U.S. Pat. No. 11,351,514, which is herein incorporated by reference.

In the present context, the microgels described herein may be considered particles (or the like) that may be formed by the systems/apparatuses of said patent(s). Moreover, materials described in said patent may be used in combination with components described herein, in various approaches.

The PIADE processes described herein overcome limitations of prior attempts to form microgels by traditional microfluidic fabrication methods, which suffer from low throughput, low yield, and limited usable biomaterials. In some approaches, a PIADE process fabricates micron-scale droplets via an aerodynamically shearing air jet and/or acoustic vibration force that causes droplet formation up to two orders of magnitude faster (e.g., 1 kHz) than microfluidics (0.1 kHz), eliminates harsh washing steps thereby decreasing material loss from ˜80% to <5% of final product, and enables the use of a wider range of biomaterials that are 10×-100× more viscous to be processed than microfluidics, which is key to achieving higher stiffness microgels.

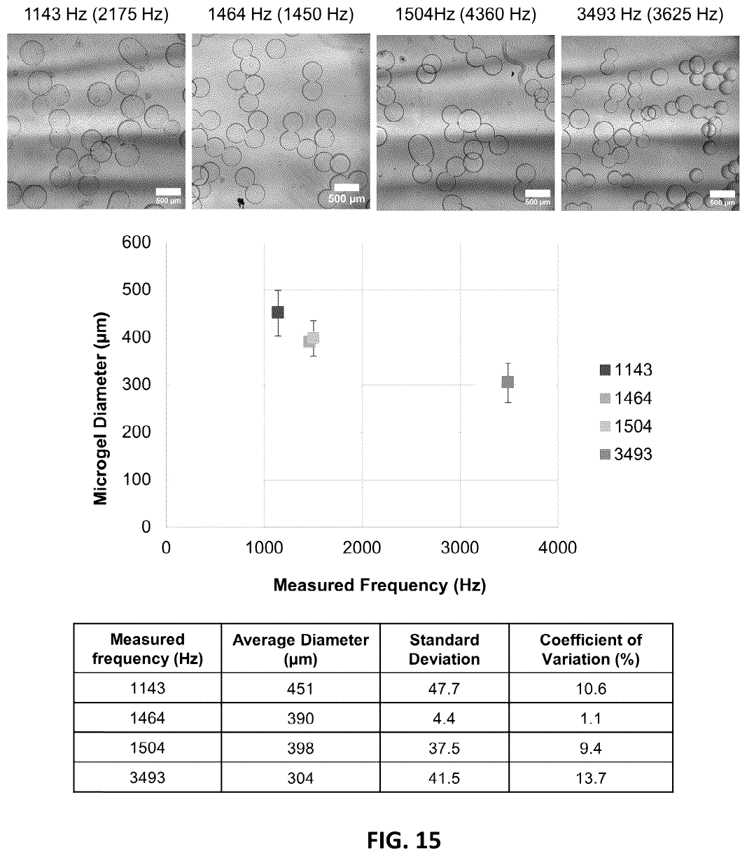

Parameters that affect droplet size, and thus the resulting microgel size, include nozzle size, vibration frequency and intensity (amplitude), and flow rate of the mixture being extruded. In general, larger nozzle sizes at given vibration frequency and flow rates correlate with larger droplets. However, increasing the vibration frequency generally correlates with smaller droplets. Higher flow rates of mixture through the nozzle generally correlate with larger droplet sizes. FIG. 15 depicts experimental results demonstrating the effect of vibration frequency on microgel diameter using the same fluid. Particularly, FIG. 15 depicts images of microgels formed at the frequency shown in Hertz (Hz), and charts depicting microgel diameter distribution and average diameters at various frequencies.

Referring again to FIG. 1, the mixture 114 from which the droplets 105 are formed may include one or more active components mixed with a photocurable resin, where the photocurable resin typically includes a polymer and a photoinitiator, and in some approaches, additional additives. The active component is selected to provide some desired objective and/or functionality in a microgel formed from the mixture 114, and/or in final product formed from such microgels.

The active component may include one or more types of live cells and/or one or more types of other component listed and/or implied herein.

In one approach, the mixture 114 from which the droplets 105 are formed generally includes live cells as the active component, which is combined with a photocurable resin at a desired concentration. Other and/or alternate active components may be present in the mixture, as described elsewhere herein.

While much of the description herein makes reference to live cells as the active component in various exemplary approaches, this has been done by way of example only. The present description is intended to enable one skilled in the art to apply the teachings herein to any active component described herein, whether including live cells or not, as well as to combinations of active components, such as those mentioned in the following paragraphs.

Preferred embodiments of the mixture 114 have one or more active components comprised of one or more types of live cells. In some approaches, the live cells may be prokaryotic cells such as bacteria. In other approaches, the live cells may be eukaryotic cells such as mammalian cells, fungal cells, algal cells, plant cells, etc. In yet other approaches, the active component may be another type of biological component, such as viruses.

In further approaches, the live cells may include a combination of two or more different types of any combination of: prokaryotic cells, eukaryotic cells, and/or other types of active component.

Illustrative live cells include, but are not limited to, live mammalian cells, live stem cells, etc. Any type of live mammalian cell may be used, e.g., stem cells, endothelial cells, hepatocytes, cardiomyocytes, pancreatic islets, fibroblasts, cartilage cells (chondrocytes), immune cells, cancer cells, etc.

Some approaches include one or more microbial components as an active component. Illustrative microbial components include bacterial cells or components, fungal cells or components, etc. Such microbial components may be considered live cells in some cases.

Some approaches include, as an active component, one or more other biological components. Illustrative other biological components include DNA, RNA, mRNA, viral components, proteins, peptides, antibodies, living-cell produced catalysts, enzymes, non-living cells, a nucleic acid-based material, therapeutic agents, etc.

In some approaches, the active component may be a cell that exhibits an optical response to contact with and/or reaction with a target substance or object. For example, a cell that produces fluorescence or luminescence in response to contacting the target substance.

Any type of combination of the foregoing live cells, microbial components and/or biological components may be present in a particular microgel, according to a plethora of approaches. Thus, any desired composition of microgel can be created.

An active component may be obtained from any suitable source. For example, the active component may be obtained from live cells, mammalian sources, algal cells, plant cells, microbial cells, etc.

The concentration of active component (e.g., cell concentration) in the resin-active component mixture may be in any desired range. In general, the concentration of active components in the microgels may correlate to a Poisson distribution, as well as is dependent to some extent on the size of the microgels.

At the lower end of said range, the cell concentration (and/or equivalently, other component concentration) of a mixture having live cells may be very low, e.g., such that some of the formed microgels have two or three cells therein, others have a single cell therein, and perhaps some of the formed microgels having no cells therein. At a higher end of said range, the cell concentration may be up to an amount that still allows formation of microgels of the desired characteristics, e.g., size, stiffness, etc.; allows extrusion; etc. For example, a range of concentration for most living cells in a resin-cell mixture is from <100 cell per mL of resin up to about 1×10⁹ cells per mL of the resin-cell mixture, any subrange in between, and possibly higher in some cases (e.g., the upper limit may be higher for bacteria, which are typically smaller in size). In general, the upper limit of cells in a resin-cell mixture may be defined by a propensity of the mixture to clog the nozzle of the PIADE apparatus, which can be determined via routine experimentation, as would become apparent to one skilled in the art after being apprised of the present disclosure.

Note that live cell viability and function can be concentration dependent where some cells fare better when they are exposed to signaling from other cells and/or are exposed to secreted factors from adjacent cells while others fare better at lower concentrations. For example, using stem cells as exemplary, cell density of mesenchymal stem cells in microgels has been shown to influence their capacity to differentiate to bone cells. Other studies have shown that a higher concentration of mesenchymal stem cells lead to higher secretion of proangiogenic cytokines for vascular repair and tissue regeneration. Here, the density of cell-hydrogel interactions may have played a role in cell differentiation and behavior. The desired cell density within the microgels is also dependent on the end use of the cell-embedded microgels. For example, for recovery of rare earth metals from electronic wastes it has been shown that a high loading density of microbes within microgels is preferable. However, in the case of cell sorting and selection, it may be desirable to encapsulate single cells. Accordingly, for biological applications, it may be preferred to use mixtures having a concentration of live cells therein, such as >1×10² cell per mL.

The polymer component of the mixture may include any monomer, polymer, copolymer, etc. or blend thereof that results in a mixture which can be crosslinked to form microgels having the desired characteristics and composition. For example, the polymer component of the mixture may include any suitable photocurable material, blend of photocurable materials, or blend of photocurable and non-photocurable materials. Examples of such polymer components include synthetic polymers such as polyethylene glycol (PEG). Further, within the potential PEG-based polymers, various types of PEG-based chemistries may be used, such as polymers based on acrylate-based chemistries for PEG, polymers based on thiol-ene based chemistries for PEG, etc. Regarding the non-photocurable materials, a material that provides some desirable effect may be mixed into the resin. Because the material does not crosslink to the polymer matrix, such material may tend to leach out of the polymer matrix over time, thereby providing a time-dependent effect, e.g., of feeding a nutrient to the cell over time.

Natural polymer components may be used. Examples of natural polymer components include gelatin, hyaluronic acid, fibrin, collagen, chondroitin sulfate, alginate, agarose, etc. Synthetic and natural materials may be modified with cell adhesive peptides or degradable crosslinkers, etc.

In general, it is desirable to select polymer components that are not significantly detrimental to the viability of the live cell and/or other active component in the mixture nor in the microgel. Moreover, material chemistry and composition will affect the mechanical properties (stiffness, porosity, etc.) of the cured microgel and therefore diffusion of nutrients to cells after encapsulation. Fabrication parameters such as UV intensity and exposure (or dosage) will also play a role in microgel properties. See, e.g., FIG. 15 and related description. In some approaches for cell encapsulation, a balance may be struck between rapid in-air curing while maintaining microgel material properties (stiffness, porosity, etc.) conducive to cell health.

Degradability of the polymer matrix of the microgel may be desirable, such as when it is desired that the polymer matrix degrades for some reason. For example, this may allow the cells to remodel the environment and produce their own extracellular matrix (ECM). In some approaches, a degradable cross linker may be added to the polymer mixture. Cell secreted factors may also degrade the polymer matrix. Immune cells, e.g., of a host's body, may also degrade the polymer matrix, when the microgels are injected into a patient's body or a crosslinked scaffold is implanted into a patient's body. Degradability of the polymer matrix should be balanced with stability of the material, to thereby allow the cells to perform their desired function, without the polymer matrix dissolving too early. Thus, the microgels may provide an initial structure for cells, as well as provide initial components needed by the cells to remain healthy and maintain their phenotype, while over time allowing the cells to remodel the environment and create mature tissue.

In some approaches, the polymer component is configured to create a matrix that enables a time release of some other component, such as a nutrient or a cell, via leaching out, diffusion via pores of the polymer matrix, dissolution or degradation of the polymer matrix, etc. In another approach, the polymer component is configured to create a matrix that enables a triggered release of some other component, such as a nutrient or a cell, upon being exposed to some trigger such as a particular pH, temperature, ionic concentration, enzyme, or other internal or external stimuli.

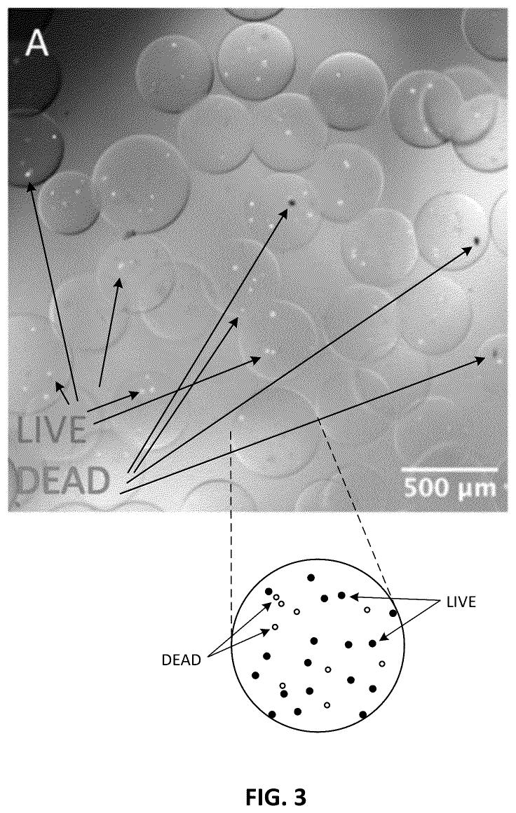

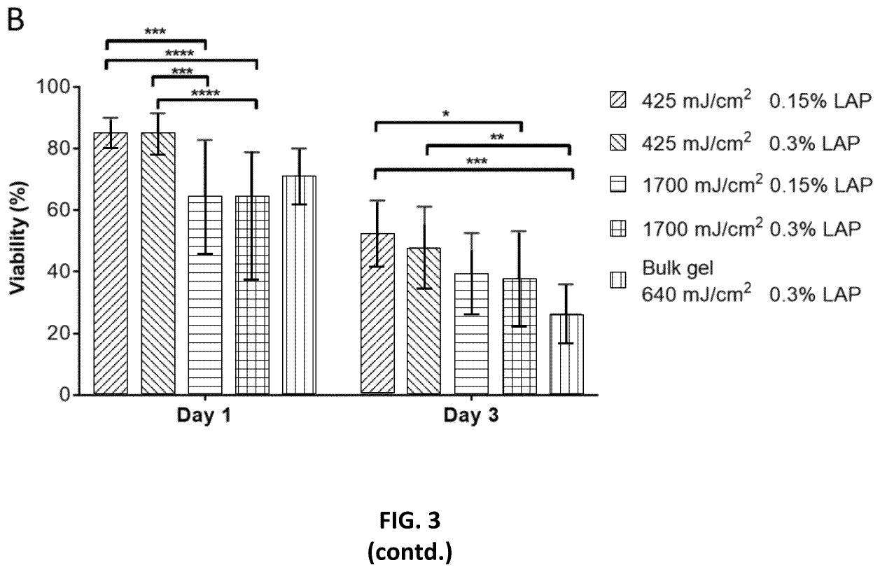

In FIG. 3 (described below), a material using 5% PEG norbornene (8 arm, 20 kDa molecular weight) and 5% PEG thiol (3.5 kDa) with 0.3% photoinitiator was used for cell encapsulation.

The photoinitiator component of the mixture may be any suitable photoinitiator or combination of photoinitiators. Generally, the photoinitiator used may be one that is selected based on the polymer component and/or the light source. For example, if a light source having a wavelength of 365 nm is used, a photoinitiator compatible with this operable at 365 nm may be selected. Known photoinitiators may be used.

A preferred photoinitiator in a given approach is a biocompatible photoinitiator compatible with the active component, i.e., that does not significantly decrease viability or activity or destroy the active component, e.g., preferably causing less than 10% loss of viability or activity of the active component, and ideally causing essentially no viability or activity nor destruction of the active component. For example, some photoinitiators may create radicals that can cause radical damage to live cells, significantly decreasing their viability.

In FIG. 3 (described below), the photoinitiator used was lithium phenyl-2,4,6-trimethylbenzoylphosphinate (LAP) because it has peak absorbance at 365 nm.

One or more additives may be present in the resin mixture. Exemplary additives are presented in the next few paragraphs. A given additive is preferably present in an effective amount to promote the desired purpose for their addition.

In one approach, a radical scavenger may be added. Viability of live cells is influenced by the time that the cells are in the uncrosslinked resin and their exposure to radical species generated during the photopolymerization process. To mitigate these effects, one or more radical scavengers may be incorporated into the resin to prevent radical damage of the cells. Such radical scavenger(s) may be added in an effective amount to provide a desired extent of mitigation.

In another approach, a growth factor may be added, e.g., to help the cell maintain a desired phenotype, e.g., to promote cell function in a way that is beneficial to the desired objective of use of the cell. Encapsulating growth factors can also be used to direct cell growth and migration in the resulting tissue scaffold once the cell-laden microgels have been crosslinked together. The particular growth factor used may be dependent on the type of cell. For instance, for endothelial cells, vascular endothelial growth factor (VEGF) may be added to promote proliferation. For chondrocytes, transforming growth factor beta (TGF-β) and bone morphogenic proteins (BMP) assist in maintaining the healthy chondrocyte phenotype and synthesis of extracellular matrix. Growth factors may be selected to promote cell growth, proliferation, differentiation, migration, formation of vasculature, synthesis of extracellular matrix, etc.

Further approaches include nutrients that promote viability and/or proliferation of live cells. Illustrative nutrients include glucose, salts, minerals, amino acids, vitamins, etc.

Some approaches may add ECM or ECM components, such as collagens, proteoglycans, elastin, fibronectin, laminins, glycosaminoglycans such as heparin, and other glycoproteins.

Further approaches may include a viscosity modifier to modify the viscosity of the resin which influences microgel or capsule fabrication. Yet other approaches may include a pH modifier. Further approaches may include an oxygen source to promote viability.

In some approaches, a coloring agent of known type may be added to the mixture to impart a color on the microgels. For example, microgels of different types can be given different colors, which may be useful for visualizing placement of different types of microgels having different colors in a particular composite structure, etc. Similarly, a fluorescent material may be added to the mixture to impart fluorescence on the microgels formed therefrom.

In yet other approaches, additives of known type that change color, fluorescence, or luminescence and/or become colored, fluorescent, and/or luminescent upon reacting with and/or coming into contact with some target material or object in the environment may be added to the mixture, thereby creating microgels that provide an optical response indicating presence of the target material or object.

In some approaches, a fluorescent dye may be used to track pH, oxygen, etc. For example, fluorescein isothiocyanate (FITC) exhibits increased fluorescence with pH, and thus may be added as a pH indicator.

The formed microgels thus include the live cells and/or other active component(s) encapsulated in the crosslinked polymer matrix. Note that some of the live cells and/or other components may also be present at an outer surface of the microgel, which can be useful in directing cell fate as well as biomarker and/or biosensing applications.

FIG. 3, part A, is an image of microgels having 4T1 mammary carcinoma cells, after one day of encapsulation, where the microgels were formed via photopolymerized IADE process during experimentation to study mammalian cell viability. The representation of an expanded microgel in Part A is presented by way of illustration to show how cells embedded within a microgel might be distributed and does not represent the actual composition of the microgel purported to be shown thereby.

FIG. 3, part B, is a chart depicting the viability of live cells in microgels formed during the experiment with different UV lamp intensities. For this demonstration, the inventors utilized a cell concentration of 3.0×10⁵ cells/mL, an applied fluid pressure of 35 psi (800 μL/min), a vibration frequency of 1450 Hz, and a 100 μm nozzle diameter. Cell viability was analyzed 1 and 3 days after encapsulation using a commercially available live/dead staining kit that utilizes calcein AM (live cells-green) and ethidium homodimer-1 (dead cells-magenta). One day after encapsulation, cells in microgels fabricated with 425 mJ/cm² had a significantly higher viability than those fabricated with 1700 mJ/cm² for both 0.15% (85±5%, 64±18%) and 0.3% photoinitiator (85±7%, 58±21%). The means of the microgel samples were not significantly different than from a bulk hydrogel. On day 3, the differences in cell viability became more distinct and all conditions were significantly lower from their day 1 counterparts. This decrease in viability is most likely due to free radicals generated during photopolymerization and the use of UV light. In addition, the combined effect of shear stress generated from nozzles and the time cells spent in the uncured resin can play a role in lowering cell viability relative to controls. As exemplified in part B, these results show that using lower UV dosage during PIADE fabrication leads to higher cell viability in the cell-laden microgels. Despite experiencing similar UV dose and photoinitiator concentration, microgels fabricated with 425 mJ/cm² showed significantly higher cell viability (0.15%-52±11%, 0.3%-48±13%) than the bulk gels (0.3%-26±10%) on day 3. While not significant, microgel viability for 1700 mJ/cm² and 0.3% photoinitiator had a higher mean viability (38±13%) than bulk gels (26±10%) despite experiencing an order of magnitude higher UV dose which suggests that microgels likely allow greater diffusion of nutrients and oxygen to the cells than the bulk gels because of their higher surface area to volume ratio.

To reduce the time that live cells are present in uncrosslinked resin, some approaches mix the live cells with the uncrosslinked resin soon or immediately before the resulting mixture is jetted from the nozzle. Any technique that would become apparent to one skilled in the art after reading the present disclosure may be used.

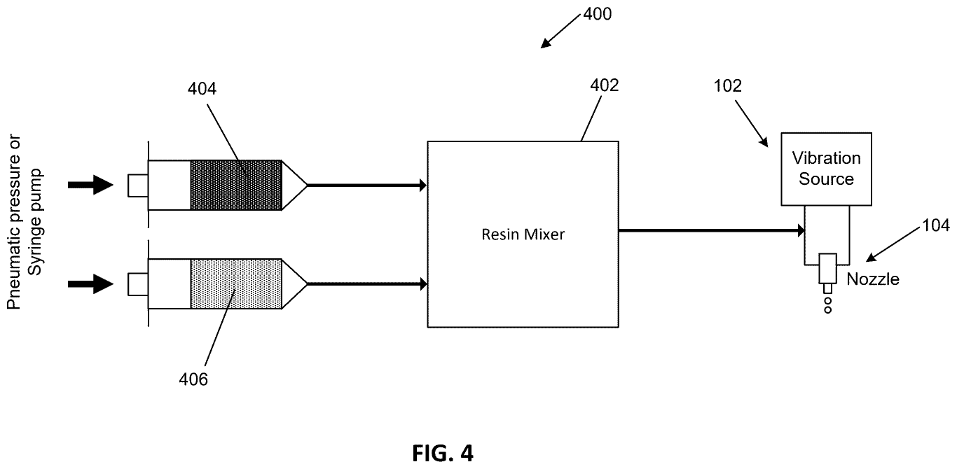

FIG. 4 depicts a PIADE system 400 in accordance with one approach. As an option, the present system 400 may be implemented in conjunction with features from any other embodiment listed herein, such as those described with reference to the other FIGS. Of course, however, such system 400 and others presented herein may be used in various applications and/or in permutations which may or may not be specifically described in the illustrative embodiments listed herein. Further, the system 400 presented herein may be used in any desired environment.

The system 400 includes a resin mixer 402 positioned to mix a resin 404 with live cells 406 on demand, just before the resulting mixture is provided to the nozzle 104. Other components may be similar to those presented in other systems described herein, e.g., as in FIGS. 1 and 2.

With continued reference to FIG. 4, the system 400 may employ any of a variety of passive or active mixing strategies of known type, that ensure the resin components are mixed at the correct proportions to reach the desired target final resin. Moreover, the system 400 may have multiple separate reservoirs for different resin precursor solutions or components thereof, to allow formation a plurality of resin combinations in real time, e.g., by changing the amount of precursor/component supplied from each reservoir. Likewise and/or alternatively, the system 400 may have multiple separate reservoirs for different active components (e.g., types of live cells) to allow formation a plurality of different mixtures in real time, e.g., having different compositions, different cell concentrations, gradients in deposited microgels, etc. Thus, the composition of the microgels being formed may be changed at least once during a continuous formation process in which a desired amount of microgels are formed before pausing or terminating the formation process.

In some cases, the polymer component may be harmful to cells when the polymer component is in the un-crosslinked state. To minimize damage to the cells, the components may be combined in the resin mixer 402 just prior to droplet formation.

Microgels, in accordance with various approaches, may contain different phases within the microgels, e.g., in the form of layered structures that contain one to multiple cores. Core-shell microcapsules can be made using concentric nozzles, for example. Microgels, in accordance with various approaches, may have structures that are blended with dissolved or emulsified smaller domains. Thus, in various approaches, live mammalian cells may be encapsulated in solid microgels. However, many different and/or alternate cell types (mammalian, bacterial, fungal, plant, algal) may be encapsulated in both solid microgels and core-shell microcapsules.

In various approaches, the microgels may be generally spherical, oval-shaped, and/or irregularly shaped.

A general average size range of the microgels (average diameter) may be in a range of about 1 μm to about 3 mm, or in any subrange within this range. In some approaches, the average size range is 1 μm to about 400 μm. For mammalian cell encapsulation, a preferred size range, in some approaches, may be about 50 μm to about 200 μm.

As noted herein the size of the microgels produced is tunable, e.g., by adjusting parameters of the fabrication process and/or of the mixture. Accordingly, not only can microgels of particular average size be formed, but microgels of differing size may be formed, e.g., to achieve a particular size distribution, to form microgels of differing composition and size, etc.

The microgels may be used for a plethora of possible applications, according to various aspects of the present invention. As described in more detail below, microgels may be used to form composite structures, such as scaffolds; biosensors; etc.

In some approaches, cell laden microgels may be frozen for long term storage until ready to use. Frozen microgels and/or objects formed with said microgels may be shipped and sold commercially.

In various approaches, microgels are stacked and crosslinked (coupled together) into a gradient tissue scaffold.

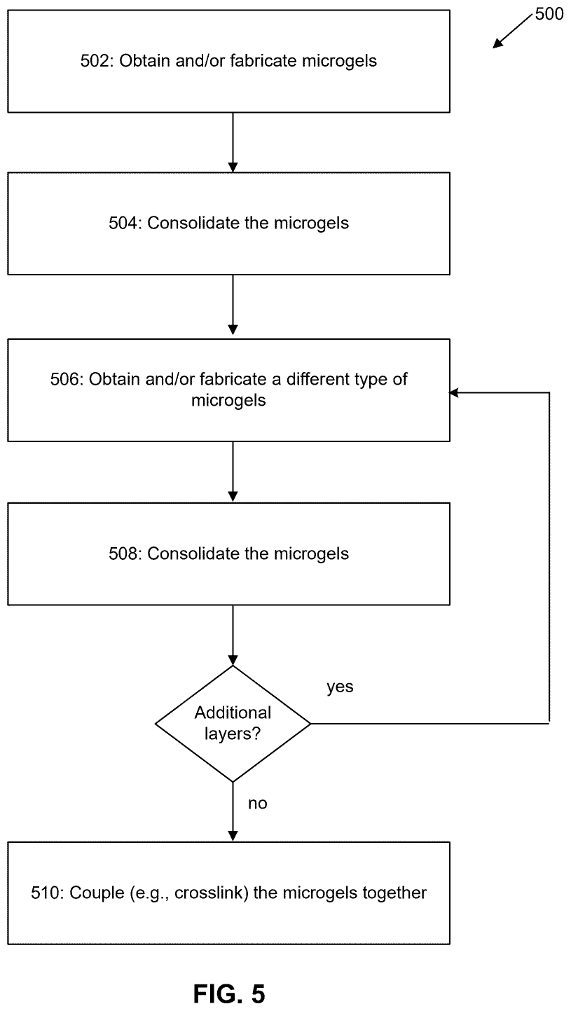

FIG. 5 shows a method 500 for forming composite structures from microgels, in accordance with one aspect of the invention. As an option, the present method 500 may be implemented to construct structures such as those shown in the other FIGS. described herein. Of course, however, this method 500 and others presented herein may be used to form structures for a wide variety of devices and/or purposes which may or may not be related to the illustrative embodiments listed herein. Further, the methods presented herein may be carried out in any desired environment. Moreover, more or less operations than those shown in FIG. 5 may be included in method 500, according to various embodiments. It should also be noted that any of the aforementioned features may be used in any of the embodiments described in accordance with the various methods.

In operation 502, microgels are obtained and/or fabricated.

In operation 504, the microgels may be consolidated, e.g., caused to become positioned closely together in a container. For example, if the microgels are in a fluid, a consolidation technique such as centrifugation may be performed.

In another approach, instead of depositing microgels onto the container while in buffer or media and then centrifuging, media may be removed from the microgels and then the microgels can be stacked directly on top of one another, e.g., by pipetting, dispensing, or any other suitable technique.

The method 500 may jump to operation 510 if no more layers are to be formed. If one or more layers of additional types of microgels are to be formed, the method proceeds to operation 506.

Operation 506 includes obtaining and/or fabricating a different type of microgels, and depositing the different type of microgels above the consolidated microgels.

In operation 508, the different type of microgels are consolidated above the previously-consolidated microgels, e.g., by centrifugation. The different type of microgels may be different from at least some of the microgels therebelow in any respect, e.g., active component, porosity, composition, size, stiffness, etc.

Operations 506 and 508 may be repeated for additional types of microgels to form additional layers or sections of microgels until the basic pre-crosslinked configuration of the composite structure is created. Note that all layers of the structure formed thus far may have different types of microgels. In other approaches, some of the layers may have the same microgels therein, though separated by one or more layers of other types of microgels.

Note that operations 502 and/or 506 may also include blending two or more diverse types of microgels together. For example, two types of microgels not yet present in the structure may be combined. In another approach, the different type of microgel added in operation 506 may be blended with the previous type of microgel. Further, gradients in microgel composition of the layer being formed may be created by changing the mixing ratio of the two or more types of microgels during deposition thereof.

Further, in some approaches, sharp transitions and/or gradients may be formed within particular layers of microgels by causing a change in some characteristic of the layer, e.g., by positioning one type of microgel in one part of a layer, and another type of microgel laterally thereto in another part of the layer; forming the layer from one side to the other while changing a ratio of first microgels to second microgels of different composition, porosity, stiffness, etc. as the layer is constructed thereacross; etc. Any suitable technique may be used to create the transitions/gradients. Examples include pipetting and/or strategic dispensing of the microgels.

In some approaches, the porosity within the stack of microgels, e.g., between adjacent microgels, may be controlled by selecting the sizes of the microgels in a particular region of the stack. For example, use of larger microgels provides a region with larger porosity and lower density than a region formed from smaller microgels of the same composition. Porosity may also be controlled by varying the parameters used to stack the microgels such as centrifugation speed and duration. The porosity in turn may affect how cells migrate through the stacked construct, how they interact with one another, etc. How the cells behave may be a function of such migration and/or interaction, i.e., the cells may behave differently in different stacked constructs having different porosities, e.g., the cells may secrete different proteins or ECM depending on the porosity and/or density.

Note that in some approaches, the microgels may not need consolidation, and thus operation 504 and/or 508 may be omitted, at least for some of the types of microgels.

In operation 510, the structure thus formed is crosslinked or otherwise processed to couple the microgels together, thereby forming the composite structure. Any coupling process may be used, with crosslinking being a preferred coupling mechanism. Any additive to promote coupling that would become apparent to one skilled in the art after reading the present disclosure may be used. In a preferred approach, the coupling step includes illuminating the structure with light to induce crosslinking. In one approach, the microgels may be caused to crosslink with one another. In another approach, an additive is placed in voids between the microgels, and the additive is caused to crosslink to the microgels, thereby linking the microgels together.

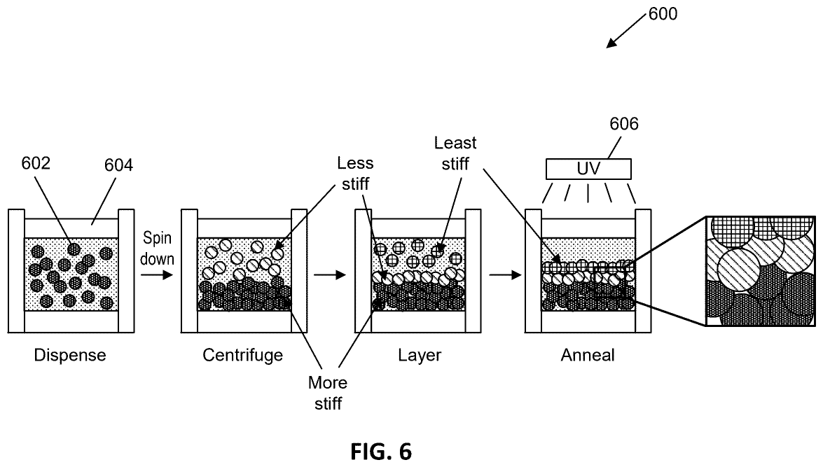

FIG. 6 graphically depicts a method 600 for assembling microgels layer-by-layer to make a porous gradient tissue scaffold, in accordance with an experiment conducted in according to one aspect of the invention. As an option, the present method 600 may be implemented to construct structures such as those shown in the other FIGS. described herein. Of course, however, this method 600 and others presented herein may be used to form structures for a wide variety of devices and/or purposes which may or may not be related to the illustrative embodiments listed herein. Further, the methods presented herein may be carried out in any desired environment. Moreover, more or less operations than those shown in FIG. 6 may be included in method 600, according to various embodiments. It should also be noted that any of the aforementioned features may be used in any of the embodiments described in accordance with the various methods.

In the illustrative approach of FIG. 6, live cells (and/or other active component(s)) were encapsulated in microgels.

Three types of microgels were formed, each having a different cell density and stiffness relative to the other types. The microgels were assembled layer-by-layer to make a porous gradient tissue scaffold.

As shown, the tissue scaffold was constructed on a hanging Transwell insert. Microgels 602 were dispensed and centrifuged for 1 min at 150 rcf on an Eppendorf 5804R with A-2-DWP rotor with acceleration (0.852 rcf/sec) and deceleration (0.794 rcf/sec) levels=1. The microgels in this example were collected in a liquid 604 comprising their cell culture media, so that the cells had the nutrients they needed to survive.

The collection and centrifugation steps were repeated with the additional types of microgels until the desired number of layers were achieved.

A coupling (e.g., crosslinking) solution was then dispensed on top of the stacked microgels and centrifuged at the same settings to allow a thin layer of solution to coat the microgels. The microgels were then coupled (e.g., crosslinked) together by exposing the stack of layers to a 32 μW/cm², 365 nm light source 606 for 3 minutes.

In this example, the crosslinker solution included 2% PEG norbornene (8 arm, 20 kDa molecular weight) and 0.3% LAP (photoinitiator) because the chemistry used to make the particular microgels used here leaves excess thiol groups on the surface of the microgels that can then react to form covalent bonds with the norbornene groups in the crosslinking solution. Note, however, that other chemistries may be used to generate microgels and therefore other and/or additional crosslinkers that contain thiol, vinylsulfone, norbornene, and/or acrylate groups with or without photoinitiator may be used to couple the microgels together.

In other approaches, the microgels are not photocured together, but rather some other procedure is used to effect the coupling. For example, a pH modifier (e.g., acid or base) may be used to induce crosslinking where the chemistry of the polymer matrix is amenable to crosslinking at a certain pH. In another approach, a known clotting enzyme such as FXIII may be used as a crosslinker.

While a Transwell multiwell plate was used to stack and couple the microgels together in the experiment represented in FIG. 6, microgels can be stacked layer-by-layer in molds of different sizes or shapes.

In one aspect of the present invention, the layered structure may be created directly from the PIADE system. For example, the nozzle may be manipulated to direct microgels to certain locations in the microgel collector. A relative movement between the nozzle and collector may be used to selectively position microgels in the collector. Moreover, the composition of the microgels may be adjusted in real time to create transitions, gradients, layers, etc. of microgels having differing compositions and/or properties.

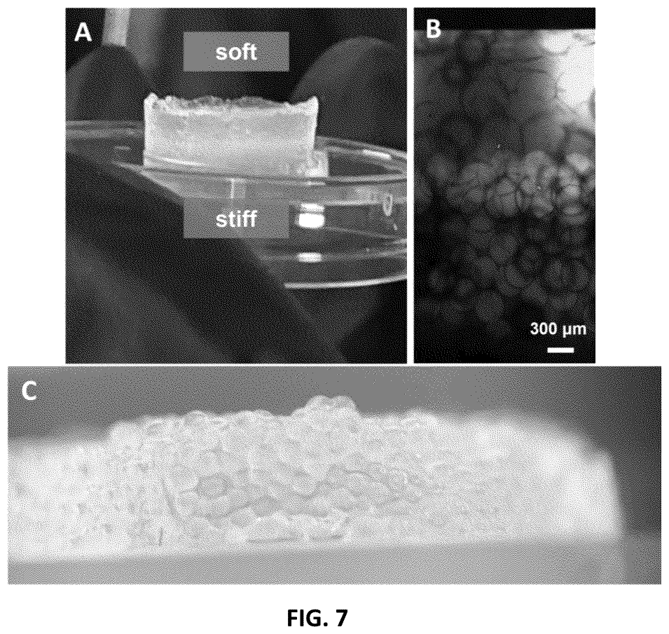

FIG. 7 depicts the stacked and coupled microgels formed during the experiment of FIG. 6. Part A of FIG. 7 is a macro view of the microgel scaffold with layered soft and stiff microgels. Part B of FIG. 7 is a microscope image of the scaffold after being cut and laid on its side, showing the distinct layers of microgels. Part C of FIG. 7 is a close up of microgels stacked within a coupled construct.

Using the stacking and coupling methodology described with reference to FIGS. 5-6 and elsewhere herein, microgels of varying sizes can be used to generate constructs with gradients in porosity, stiff and soft microgels may be used to generate tissue scaffolds with gradients in stiffness, different concentrations of molecules can be encapsulated to generate chemical gradients, the number of cells encapsulated can be controlled to generate gradients in cell density, etc.

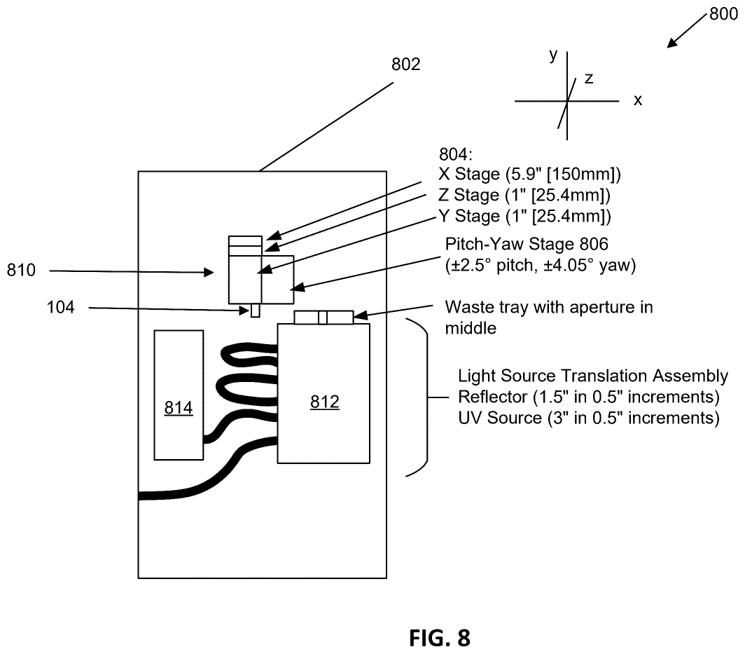

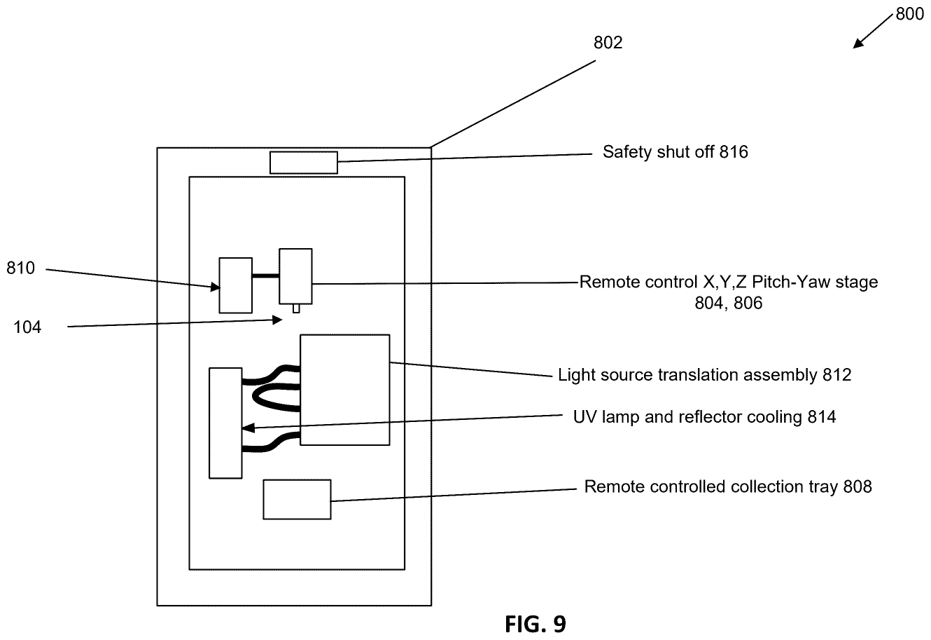

Further aspects of the present invention relate to a PIADE system, which is particularly useful for biological encapsulation. FIGS. 8-9 depict a PIADE system 800 in accordance with one approach. As an option, the present system 800 may be implemented in conjunction with features from any other embodiment listed herein, such as those described with reference to the other FIGS., especially FIGS. 1-2 and 4. Of course, however, such system 800 and others presented herein may be used in various applications and/or in permutations which may or may not be specifically described in the illustrative embodiments listed herein. Further, the system 800 presented herein may be used in any desired environment.

The system 800 shown in FIGS. 8-9 improves upon the PIADE systems of FIGS. 1-2 in several ways. Note that in various approaches, one or more of the following improvements are present in a given system 800. Said another way, the system 800 need not have each and every improvement listed herein; rather, various approaches may have different combinations of said improvements.

In some approaches, a biocontainment enclosure 802 is present to surround the nozzle/light source/receptacle or other object that receives the cured microgels. The biocontainment enclosure is configured to protect the user from the live cells and/or light; and/or protect the process from contamination, e.g., to maintain sterility of the system during formation of microgels. The biocontainment enclosure may have any suitable construction, and may be constructed at least in part of conventional materials. Preferably, at least one door is provided to allow access to the interior of the biocontainment enclosure.

One or more remote controlled features are provided in some aspects of the present invention. The remote-controlled features may provide one or more benefits, such as increased control and reproducibility of the formation process, maintaining sterility within the enclosure by minimizing handling of components of the system, safety to a human operator, etc.

The remote-control components, such as positioning hardware, control systems, wiring, etc. may be assembled from conventional parts, in a manner that renders the remote-control components compatible with the system 800. For example, positioning hardware such as actuators, worm screws, piezos, etc. may be used for positioning. A computer, control circuit, chip, etc. may be used to control the positioning hardware. Controllers may be present to enable an operator to manually control movement of the positioning hardware.

Moreover, a 3D model or mold may be used to control positioning and/or composition of the microgels being produced, e.g., to create gradients, transitions, etc. of the compositions and/or properties of the formed layers of the microgels. For example, a 3D model or mold of a cartilage structure may be used to control formation of a scaffold of microgels that has properties and cell types mimicking human cartilage.

In some approaches, remote controlled linear stages 804 allow precise positioning of the nozzle in relation to the light source, thereby enabling such things as more reproducible bead production. Shown are X, Y and Z stages that provide positioning of the nozzle in the X, Y and Z directions. The light source may be a UV lamp or other suitable light source. For example, blue light at 405 nm tends to be more cell friendly than other wavelengths, and thus may lead to preserved viability.

A UV lamp is used in this description by way of example only. The stages 804 may be constructed, at least in part, of conventional components.

Note also that if longer cure times are needed/desired (e.g. lower UV intensity or alternate cure methods are required for cell viability which necessitate longer cure time to achieve a fully cured microgel), this can be achieved a number of ways including but not limited to stacking multiple lamps on top of each other to increase the fall height and resident time in the curing zone and/or adding a secondary cure stage after collection. Further approaches to control curing may include pulsing the UV lamp, control over zones of UV exposure (e.g., by controlling several lamps independently, using shutters or screens to partially block the light, etc.), etc.

In some approaches, a remote-controlled pitch-yaw stage 806 enables fine adjustment of the fluid jet angle relative to the UV lamp, allowing the user to compensate for variance in jet direction resulting from differences between nozzle assemblies and/or flow parameters. The stage 806 may be constructed, at least in part, of conventional components.

In some approaches, a remote-controlled relative movement system may provide relative movement between the nozzle and a collection area of the system (e.g., a container for collecting the microgels, a platform for receiving such a container, etc.). This feature allows approximate positioning the microgels in the container, e.g., to create structures with gradients, etc. by changing the composition of the microgels during formation to position microgels of differing composition at different locations in the container.

In one approach, a remote-controlled collection tray shuttle 808 enables precise, programable, and/or synchronized movement of the collection vessel (e.g., timed collection of beads and/or automated retraction when the UV lamp is disabled). The remote controlled collection tray shuttle 808 may be constructed, at least in part, of conventional components.

In another approach, the collection tray may be part of a rotary design. Rotary motion of the collection tray enables fabrication of microgels with varying parameters such as type of resin, UV intensity, flow rate, etc. to be changed without needing to open the system to change the collection container between runs. This enables such things as allowing rapid testing of different experimental conditions by implementing a rotary sample collection vessel containing one or more collection vessels, fabrication of microgels with varying parameters, etc. Experimental and/or fabrication conditions of interest may involve different droplet generation frequencies, UV powers/dosages, and different microgel compositions, especially when paired with an in-line mixer (e.g., as shown in FIG. 4). The collection vessels may be selected based on a position index allowing for one or more experimental conditions to be collected without having to stop the droplet generation sequence.

In some approaches, a remote-control system 810 that controls the nozzle enables precise, programable, and synchronized movement of the nozzle (e.g., automated retraction when the UV lamp is disabled and/or cyclical nozzle movement during bead generation). The remote-control system 810 may be constructed, at least in part, of conventional components.

The remote-control component(s) of a PIADE system assist in ensuring sterility inside the enclosure of the PIADE apparatus, which may be important for preserving the viability of the active material, for preventing introduction of contaminants and/or dangerous microorganisms that could in turn contaminate the situs of use of the resulting microgels, etc.

In some approaches, a positioning system 812 provides the ability to move the light source and/or reflector position in the z axis (up and down) and/or x axis (into and out of the enclosure) for greater adaptability. The positioning system 812 shown includes a light source translation assembly. The positioning system 812 may be constructed, at least in part, of conventional components.

In some approaches, the reflector position may also be moved independently of the lamp position in the x-axis (to increase or decrease the distance between the UV source and reflector). Reflector position affects UV dose uniformity and the location of peak UV dose.

One approach may have a mechanism to minimize bulk UV exposure at the nozzle, such as an optical shutter timed to synchronize with capsule production. One approach may use a global shutter on the lamp, etc.

In some approaches, an upper waste tray may be present within the enclosure. The waste tray may be any sort of barrier or container that is UV opaque, which rests on or above the UV lamp/reflector assembly, with a small orifice through which the generated droplets can fall, but limits the bulk amount of UV dosage the nozzle receives during system operation. The waste tray is preferably made of materials that are not prone to overheating/melting due to its proximity to the UV lamp. Preferred materials for the waste tray include metals.

Use of an upper waste tray is preferred when using materials which are rapidly curing (e.g., thiolene resin), because without the UV blocking, the resin might cure as soon as it exits the nozzle, clogging the system and stopping a run.

In some approaches, the upper waste tray includes a shutter whose frequency and duty cycle of being “open” or “closed” can be tuned to match the production frequency of the droplets. This feature would thereby minimize the exposure at the droplet generation source preventing clogging of the nozzle. The upper waste tray may, in some approaches, be fitted with a wiper assembly to help clear the orifice due to any cured material that may build up over the course of the run.

In some approaches, an active cooling system 814 is present to maintain the light source (e.g., high powered UV lamp) at a safe operating temperature and ensure cooling of the reflector. Prior IADE system would require runs at 100% UV power to stop after 15 min on known systems. The active cooling system 814 enables operation for much longer times, even when operating at 100% UV power. Moreover, heat in the region of the lamp and reflector have been found to create instabilities in droplet formation. The active cooling system reduces such instabilities.

The cooling system 814 may be constructed, at least in part, of conventional components. The cooling system may be designed to carry heat away from the reflector, to maintain about a desired temperature in the vicinity of the droplets/microgels, to maintain about a desired temperature in the interior of the enclosure 802, etc. In some approaches, the desired temperature is within a viability temperature range for the active component. For example, the cooling system may maintain a temperature within the enclosure of between about 25 and about 40 degrees Celsius, and in some approaches, between about 27 and about 37 degrees Celsius.

In a preferred approach, the cooling system 814 includes conduits through which a conventional cooling fluid such as water, ethylene glycol, water+ethylene glycol, chilled air, etc. is flowed. The conduits may be positioned on the reflector to carry heat away from the reflector.

In some approaches, the cooling system 814 may include a gas flow system for flowing cooled gas through the interior of the enclosure 802 for maintaining about a desired temperature in the interior of the enclosure 802. Care should be taken to avoid disruption of the droplet formation by turbulent air movement through the enclosure.

In some approaches, a gas flow control system may be used for various purposes, such as to filter gas flowing through the enclosure, e.g., with a HEPA filter, to purge gas in the enclosure, to exchange gas within the enclosure, etc.

In some approaches, the gas flow control system provides vacuum and/or air flow to direct microgels toward the collection vessel.

In some approaches, the gas flow system may assist in droplet formation, e.g., by creating a vacuum within the enclosure. To elaborate, for small nozzles diameters (e.g., about 100 μm or less), the required exit velocity to create a stable jet, as described elsewhere, may, and likely will, exceed the terminal velocity of small droplets. As a result, upon droplet formation, these droplets experience a negative acceleration due to drag forces, resulting in a decrease in flight velocity. This reduction in velocity can occur to such a degree that subsequently produced droplets can catch up to the previously generated droplets. If droplets meet before curing has begun, droplets can merge to form a larger coalesced droplet, leading to multi-modal microgel distributions. If droplets meet in mid-air after curing has begun, these droplets can chain together, creating caterpillar-like structures. Thus, placing the PIADE system under vacuum during operation would reduce or eliminate the effects of in-air drag causing these coalescence behavior.

Additionally, placing the interior of the enclosure under vacuum reduces the presence of convective air currents in the lamp-reflector region where air heated by the lamp rises. These non-laminar air currents can cause undesired effects on the droplet stream trajectory.

In a similar manner, a directed air flow may be generated either by positively “pushed” air flow from the top to the bottom of the PIADE system or by negatively “pulled” air flow from the bottom of the PIADE system via a vacuum pump.

In some approaches, an automatic safety shut off system 816 turns off the UV lamp when the doors of the enclosure are opened. In some approaches, the automatic safety shut off system 816 may also or alternatively turn off the UV lamp when the cooling system is not operating. The automatic safety shut off system 816 may be constructed, at least in part, of conventional components, such as a switch that is responsive to movement and/or position of one or more doors of the enclosure, an optical switch that detects when a door is open, etc.

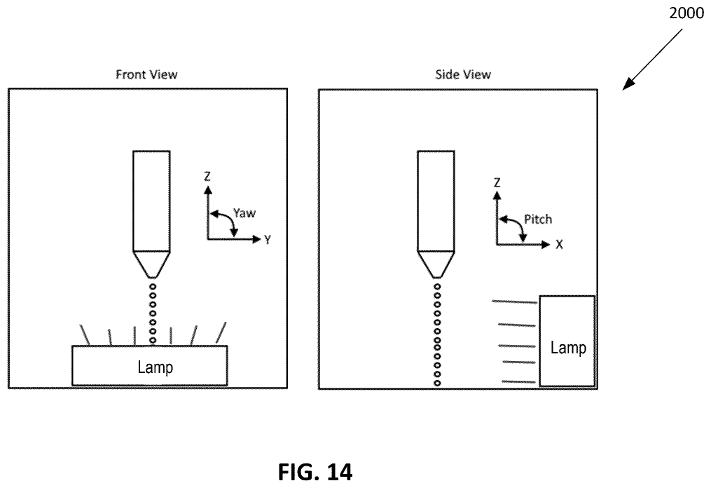

FIG. 14 depict a PIADE system 2000 in accordance with one approach. As an option, the present system 2000 may be implemented in conjunction with features from any other embodiment listed herein, such as those described with reference to the other FIGS., especially FIGS. 1-2, 4, and 8-9. Of course, however, such system 2000 and others presented herein may be used in various applications and/or in permutations which may or may not be specifically described in the illustrative embodiments listed herein. Further, the system 2000 presented herein may be used in any desired environment.

As shown in FIG. 14, integrated lighting and/or cameras may also be used to improve visualization of the nozzle during alignment, especially when nozzle alignment is done via remote control. For example, cameras positioned at right angles would allow the user to accurately determine the position of the nozzle and make appropriate adjustments as needed. The cameras may also enable remote monitoring of microgel formation.

### In Use:

Various aspects of the present invention may be used for any suitable purpose.

Exemplary uses of some aspects include in-air photopolymerization, microencapsulation, cell encapsulation, formation of microgels, formation of cell-embedded microgels, formation of microcapsules, formation of modular tissue, formation of tissue scaffold, formation of gradient tissue, formation of hydrogels, formation of biomaterials, tissue engineering, and microfluidics.

In one approach, PIADE-generated cell-embedded microgels are used to fabricate modular, gradient tissue scaffolds. For example, cartilage cells (chondrocytes) may be encapsulated into polyethylene glycol (PEG) microgels to generate a gradient cartilage tissue. Such cartilage scaffold represents the first construct to mimic the gradient nature of cartilage biomechanics.

The methodology presented herein enables introduction of more complexity into tissue scaffolds than heretofore thought possible, by generating gradients in materials, porosity, cell types, and growth factors by controlling the characteristics of the encapsulated cargo, microgel material, and microgel size.

As also mentioned above, controlled consolidation of microgels of like or different types enables fabrication of customizable living microscale constructs, opening the door to a wide variety of applications listed below:
Feedstock for customizable engineered tissue scaffolds
   Cells can be encapsulated within the microgels themselves and/or seeded into the scaffold after the microgels have been coupled together.
   The microgels can be used in combination with any of a plethora of 3D printing and/or bioprinting techniques such as direct ink writing (DIW), stereolithography (SLA), projection microstereolithography (PμSL), extrusion printing, volumetric additive manufacturing, and others. For example, the microgels described herein may be mixed with resins compatible with the various 3D printing and/or bioprinting techniques, and then the mixture printed/formed into 3D structures according to those techniques. The 3D structures so constructed may have any form factor achievable by the particular technique. Preferably, such resins are biocompatible with the active component, e.g., live cells.
Biosensors
   Embedded cells and/or the microgels themselves can be engineered to generate an optical signal (e.g., fluorescence) in the presence of a target material (e.g., pathogens, cytotoxic agents, a target molecule, etc.), thereby acting as living biosensors to rapidly screen for biological and chemical agents thereby aiding in developing countermeasures.
   The cells used may depend on the desired application. Examples include mammalian B lymphocyte to detect certain bacteria and viruses. Engineered E. coli may be used to respond to an autoinducer (signaling molecule) secreted by a pathogen called Pseudomonas aeruginosa.
   In some approaches, the porosity of the microgels can be selected to enable the target material to be detected to travel into the microgel. Moreover, the porosity may be selected to provide size exclusion of target materials that are not of interest. In other approaches, the microgel material may be functionalized to attract and/or promote interaction with the target material (e.g., inter-pore transport, contact with the microgel surface, etc.), and/or to repel materials that are not of interest.
   Other types of fluorescent and/or luminescent components may also and/or alternatively be incorporated into the microgels. Such components may be of known type, and may or may not be cell based. The types of fluorescent and/or luminescent components may depend on the target application and/or the desired molecule of detection. Examples include encapsulated glucose oxidase, horseradish peroxidase, and Amplex Red. In the presence of glucose, microgels fluoresce and signal intensity varies with concentration of glucose. Fluorescein and rhodamine may be incorporated into microgels for pH detection.
   These biosensors can be readily incorporated into tissue constructs that can be used to test and develop vaccines or other countermeasures.
Encapsulation of microbial cells (e.g., bacterial, fungal), plant cells, nucleic acids, enzymes, algae, proteins and/or for bioremediation and/or biofuel/biotherapeutic production.
   In one approach, microgels may be used in large scale bioreactors to scale up bioremediation or biotherapeutic production. A benefit of using the microgels is that there is a higher surface area to volume ratio, which in turn increases transport of nutrients, metabolites, and contaminants to the cells and protein product from the cells out to the media, making the remediation or production process more effective.
   One approach may include plant cells producing recombinant butyrylcholinesterase (BChE) as a prophylactic/therapeutic against organophosphate nerve agent poisoning.
   One approach includes microbe encapsulation for selective rare-earth recovery from electronic waste leachates.
   One aspect may include high-throughput screening of cells based on functional outputs such as secreted factors. This may enable selection of cells with beneficial properties for antibody production, biofuel production, directed evolution, etc. For example, one approach includes high-throughput sorting of microalgae with beneficial properties for biofuel production.

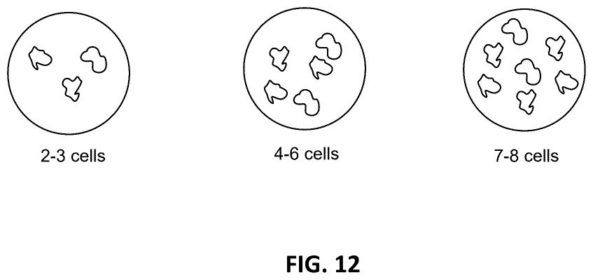

Referring to U.S. Provisional Patent Appl. No. 63/572,828, which has been incorporated by reference, FIG. 12 of said Provisional Application illustrates human skin, its gradients in tissues, and how the gradients and tissues drive its function. In some approaches, a scaffold may be constructed of microgels that mimics a section of skin, including the gradients in tissues.

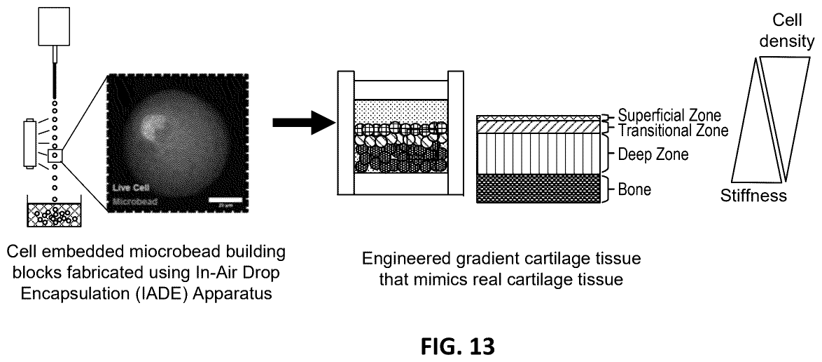

With continued reference to U.S. Provisional Patent Appl. No. 63/572,828, which has been incorporated by reference, FIG. 13 of said Provisional Application illustrates a human heart, its gradients in tissues, and how the gradients and tissues drive its function. In some approaches, a scaffold may be constructed of microgels that mimics a section of the heart, including the gradients in tissues.

With continued reference to U.S. Provisional Patent Appl. No. 63/572,828, which has been incorporated by reference, FIG. 14 of said Provisional Application illustrates cartilage, its gradients and tissues, and how the gradients and tissues drive its function. Note the gradients in stiffness and cell density.

With continued reference to U.S. Provisional Patent Appl. No. 63/572,828, which has been incorporated by reference, FIG. 15 of said Provisional Application exemplifies that some damaged tissues do not heal. For example, joint replacements currently use metal and ceramic, and because of that, they do not mimic the natural mechanical properties of cartilage. As also noted above, previous attempts at creating engineered tissues failed as in vivo replacements, in part because said attempts could not mimic the proper cell densities and/or tissue stiffness. The result of these previous attempts is that the patient's mobility remains limited, the patient is often unable to return to normal activities, and the materials wear down over time.

In some approaches, a scaffold may be constructed of microgels with live cells, where the scaffold mimics a section of cartilage, including the gradients in stiffness and cell density. Conceptually, the microgels are used as modular building blocks. The result is that the scaffold is much more likely to integrate into the existing tissue and heal.

For example, FIG. 15 of the Provisional Application depicts a tear in the cartilage (middle image, circled). A scaffold of microgels can be created in the form of a plug that fits into the tear. Moreover, a full thickness scaffold may be created to replace cartilage, e.g., if the patient has a joint with a section that no longer has cartilage.

The living cells may be sourced from any suitable source. For example, continuing with the cartilage replacement example, an autologous graft may be taken from a different portion of the joint. In another approach, stem cells may be grown to the desired cell type, e.g., chondrocyte in the case of cartilage. In yet another approach, cells may be obtained from a donor.

FIG. 12 depicts microgels of different cell loading, exemplifying the ability to create such.

The different types of microgels (size, composition, stiffness, cell loading, etc.) can be strategically positioned to provide a scaffold having regions with the desired properties, e.g., to mimic layers of tissues in cartilage. Moreover, microgels of differing size may be used in a certain region to tune porosity, stiffness, etc.

FIG. 13 is a depiction of formation of microgels having live cells therein, and the microgels being assembled together in a scaffold that mimics the physical characteristics of the layers of cartilage listed adjacent thereto. Also shown is a representation of the relative gradients of stiffness vs. cell density.

In further approaches, microgels having living cells therein may be injected at the site of a wound. Moreover, different types of microgels may be selectively injected to create a gradient in characteristics of the microgels, e.g., in terms of stiffness, cell type, etc. In one approach, human tissue (e.g., of an organ) may be partially decellularized, and microgels are injected into the decellularized region.

The inventive concepts disclosed herein have been presented by way of example to illustrate the myriad features thereof in a plurality of illustrative scenarios, embodiments, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various embodiments have been described above, it should be understood that they have been presented by way of example only, and not limitation. Thus, the breadth and scope of an embodiment of the present invention should not be limited by any of the above-described exemplary embodiments, but should be defined only in accordance with the following claims and their equivalents.

What is claimed is:

1. A live cell-laden microgel, comprising at least one live cell and a photocured resin.

2. The live cell-laden microgel of claim 1, wherein the live cell is selected from the group consisting of: a prokaryotic cell and a eukaryotic cell.

3. The live cell-laden microgel of claim 1, wherein the live cell is a live mammalian cell.

4. The live cell-laden microgel of claim 1, wherein an average diameter of the microgel is in a range of about 1 μm to about 3 mm.

5. A product, comprising a plurality of the microgels of claim 1 formed into a predefined shape.

6. The product of claim 5, wherein the product comprises the microgel and at least a second microgel having a different composition than the microgel.

7. The product of claim 6, wherein the second microgel has a different stiffness than the microgel.

8. The product of claim 6, wherein the second microgel has a different porosity than the microgel.

9. The product of claim 6, comprising a plurality of the microgels in a first layer, and a plurality of the second microgels in a second layer positioned above the first layer.

10. The product of claim 9, wherein some of the microgels in the first layer are crosslinked to some of the second microgels in the second layer.

11. The live cell-laden microgel of claim 1, wherein the microgel is an in-air drop formed microgel.

12. A method for forming a plurality of the microgels of claim 1, the method comprising:
forming the microgels via photopolymerized in-air drop encapsulation.

13. The method of claim 12, comprising changing a composition of the microgels being formed at least once during a continuous formation process.

14. An in-air drop formed microgel, comprising a component selected from the group consisting of DNA, RNA, a protein, a peptide, an antibody, a living-cell produced catalyst, a nucleic acid-based material, a prokaryotic cell, an eukaryotic cell, and a different type of biological component.

15. A product, comprising a plurality of the in-air drop formed microgels of claim 14 formed into a predefined shape.

16. The product of claim 15, wherein the product comprises the in-air drop formed microgel and a second in-air drop formed microgel having a different composition than the in-air drop formed microgel.

17. The product of claim 16, wherein the second in-air drop formed microgel has a different stiffness than the in-air drop formed microgel.

18. The product of claim 16, wherein the second in-air drop formed microgel has a different porosity than the in-air drop formed microgel.

19. The product of claim 16, comprising a plurality of the in-air drop formed microgels in a first layer, and a plurality of the second in-air drop formed microgels in a second layer positioned above the first layer.

20. The product of claim 19, wherein the layers are in the form of a scaffold having gradients in one or more characteristics selected from the group consisting of composition, porosity, cell types, and growth factors.
